(19) United States
(12) Patent Application Publication
(10) Pub. No.: US 2019/0223836 A1
(43) Pub. Date: Jul. 25, 2019

(54) ULTRASOUND DIAGNOSTIC APPARATUS AND EXTERNAL BATTERY UNIT THEREOF

(71) Applicant: Konica Minolta, Inc., Tokyo (JP)

(72) Inventors: Yuki MURAMATSU, Saitama-shi (JP); Takahiko SHIRAISHI, Tokyo (JP); Shinya NOGUCHI, Tokyo (JP)

(21) Appl. No.: 16/240,296

(22) Filed: Jan. 4, 2019

(30) Foreign Application Priority Data
Jan. 19, 2018 (JP) 2018-006820

(51) Int. Cl. A61B 8/00

(52) U.S. Cl. CPC A61B 8/4427; A61B 8/4444; A61B 8/0866; A61B 8/54; A61B 8/56

(57) ABSTRACT

An ultrasound diagnostic apparatus, including: an ultrasound diagnostic apparatus main body which transmits a drive signal to an ultrasound probe that transmits and receives ultrasound waves, and which generates an ultrasound image based on a received signal that is obtained from the ultrasound probe; and a battery unit which is externally attached to the ultrasound diagnostic apparatus main body and supplies electric power to the ultrasound diagnostic apparatus main body, wherein the battery unit includes: a battery housing which is made of resin and contains a battery therein; an electrical connector for connection to the ultrasound diagnostic apparatus main body, the electrical connector being fixed to the battery housing; and a conductive member which is arranged between the battery and an inner wall of the battery housing, and the conductive member is grounded to the ultrasound diagnostic apparatus main body.

### BACKGROUND

### Technological Field

The present invention relates to an ultrasound diagnostic apparatus and an external battery unit thereof.

### Description of the Related Art

There has been conventionally known an ultrasound diagnostic apparatus which performs, with an ultrasound probe, transmission and reception of ultrasound waves with respect to a subject under examination such as a living body, generates ultrasound image data on the basis of signals obtained from the received ultrasound waves, and displays an ultrasound image based on the data on an image display apparatus. Ultrasound image diagnosis using such an apparatus can be repeatedly performed since the state of heartbeat, movement of fetus and the like can be grasped real time with a simple operation of merely putting the ultrasound probe against the body surface of the subject, and the diagnosis is non-invasive and has a high safety.

In recent years, a portable ultrasound diagnostic apparatus which is compact and can be carried has been put into practical use, which enables diagnosis outside medical facilities such as hospitals.

In accordance with the development of portal ultrasound diagnostic apparatus, the battery drive system is adopted.

As a method for arranging the battery so as to be integrated with the ultrasound diagnostic apparatus, there is adopted an external attaching method of attaching the battery to the outer surface of the ultrasound diagnostic apparatus main body so as to be arranged outside the main body, besides an internal mounting method of arranging the battery inside the ultrasound diagnostic apparatus main body. For example, there is a method of enabling a user to use the external battery in addition to the internal battery. Since the external battery is not arranged in the internal space of the main body, the external battery has an advantage that a plurality of types of batteries having different capacities can be prepared without increasing the size of the main body.

The external battery is not internally mounted in the main body. Accordingly, it is preferable that the external battery unit has a housing (hard case) containing the battery for protecting the battery or the like. The housing makes it possible to fix, to the housing, an electrical connector for connection to the main body, and lock a fastening tool (bolt or the like) to the housing in order to surely fix the battery unit to the main body. Thus, it is easy to perform attachment/detachment and firm fixation of the external battery unit to the main body.

Metal or resin can be selected as a material of the housing of the battery unit. However, there is a tendency to require resin which is light and inexpensive in consideration of portability, low price and the like.

For example, there is a manner of mounting a plurality of batteries inside the apparatus, not limited to portable/cart type, as described in specification of US patent application publication No. 2013/0330588, and a manner of mounting a sub-battery inside an expansion apparatus such as a docking station and charging the main body battery from the sub-battery to enable extension of operation time of the apparatus as described in specifications of Japanese Patent No. 3828744 and U.S. Pat. No. 7,849,250.

However, when a plurality of batteries is mounted inside, the internal space of the main body possibly becomes tight. In a case where the housing containing the sub-battery is a metal housing, a total weight of the system increases, increasing situations in which the system cannot be carried easily.

Furthermore, in a case of producing the housing with light metal, the material is expensive and the processing cost is high, leading to a high original cost, in some cases. Thus, there are situations in which the user is required to adopt the resin housing.

However, when the housing of external battery unit is made of resin, the outer wall surrounding the battery is resin and the battery is not shielded by a metal material. Thus, it is not possible to effectively prevent electromagnetic radiation.

The resin housing of the battery unit cannot be electrically connected when contacting the ultrasound diagnostic apparatus main body since the housing is resin. Thus, the electrical connection between the battery unit and the ultrasound diagnostic apparatus main body is limited to an electrical connector.

In that case, the battery unit can be grounded only by the electrical connector, and the resin housing does not function as ground metal. Thus, the grounding of the battery unit is not sufficient, and there has been a problem in EMC (Electromagnetic Compatibility) performance. When the EMC performance is not sufficient, there can be troubles that the apparatus cannot meet the standard of EMC performance since the standard of EMC performance is strict in use of the ultrasound diagnostic apparatus, that the apparatus interferes with the operation of surrounding electronic devices, that noise is generated in the ultrasound images made by the apparatus itself, and the like.

### SUMMARY

The present invention has been made in consideration of the above problems of conventional techniques, and an object of the present invention is to enhance grounding of the battery unit, reduce electromagnetic radiation from the battery unit, and improve the EMC performance.

To achieve at least one of the abovementioned objects, according to an aspect of the present invention, an ultrasound diagnostic apparatus reflecting one aspect of the present invention includes: an ultrasound diagnostic apparatus main body which transmits a drive signal to an ultrasound probe that transmits and receives ultrasound waves, and which generates an ultrasound image based on a received signal that is obtained from the ultrasound probe; and a battery unit which is externally attached to the ultrasound diagnostic apparatus main body and supplies electric power to the ultrasound diagnostic apparatus main body, wherein the battery unit includes: a battery housing which is made of resin and contains a battery therein; an electrical connector for connection to the ultrasound diagnostic apparatus main body, the electrical connector being fixed to the battery housing; and a conductive member which is arranged between the battery and an inner wall of the battery housing, and the conductive member is grounded to the ultrasound diagnostic apparatus main body.

According to an aspect of the present invention, an external battery unit of an ultrasound diagnostic apparatus reflecting one aspect of the present invention is externally attached to an ultrasound diagnostic apparatus main body and supplies electric power to the ultrasound diagnostic apparatus main body which transmits a drive signal to an ultrasound probe that transmits and receives ultrasound waves, and which generates an ultrasound image based on a received signal that is obtained from the ultrasound probe, and the battery unit includes: a battery housing which is made of resin and contains a battery therein; an electrical connector for connection to the ultrasound diagnostic apparatus main body, the electrical connector being fixed to the battery housing; and a conductive member which is arranged between the battery and an inner wall of the battery housing, wherein the conductive member is grounded to the ultrasound diagnostic apparatus main body.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a perspective view showing an ultrasound diagnostic apparatus according to an embodiment of the present invention;

FIG. 2 is an exploded view of an external battery unit according to the embodiment of the present invention;

FIG. 3 is an exploded view of the external battery unit according to the embodiment of the present invention;

FIG. 4 is a sectional view in a state in which the battery unit is attached to the main body, according to the embodiment of the present invention;

FIG. 5 is a sectional view in a state in which the battery unit is attached to the main body, according to the embodiment of the present invention;

FIG. 6 is a graph showing frequency distribution of the electromagnetic radiation amount of an ultrasound diagnostic apparatus according to a comparative example; and

FIG. 7 is a graph showing frequency distribution of the electromagnetic radiation amount of an ultrasound diagnostic apparatus according to an example of the present invention.

### DETAILED DESCRIPTION OF THE EMBODIMENTS

Hereinafter, one or more embodiments of the present invention will be described in detail with reference to the drawings. However, the scope of the invention is not limited to the disclosed embodiments.

An embodiment according to the present invention will be described in detail with reference to the accompanying drawings.

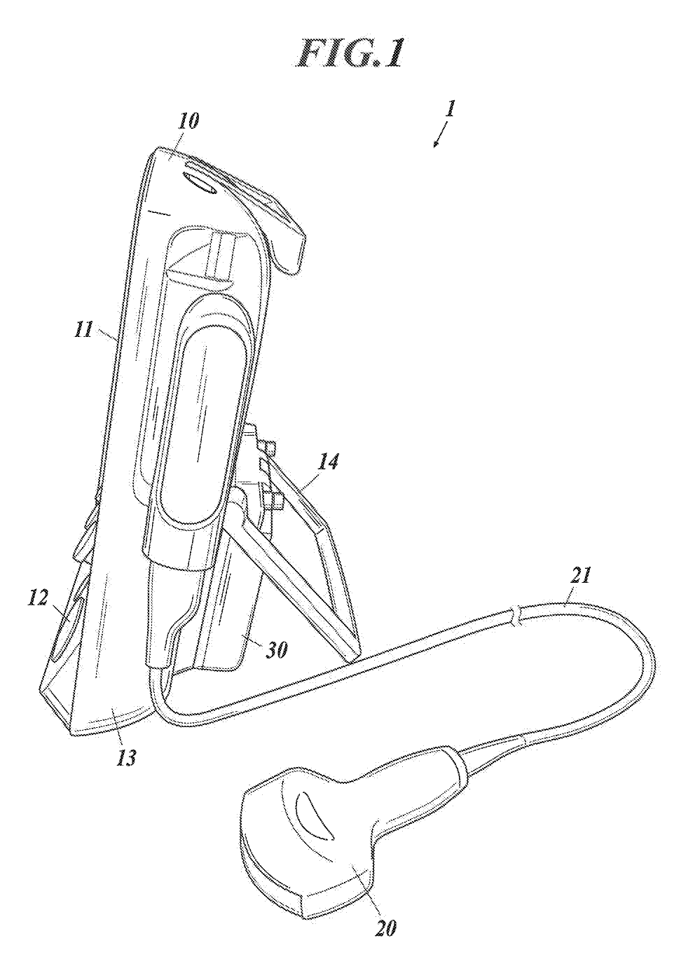

FIG. 1 illustrates a configuration example of an ultrasound diagnostic apparatus.

The ultrasound diagnostic apparatus 1 is used at a medical facility such as a hospital, and diagnoses a subject under examination such as a living body of a patient. As shown in FIG. 1, the ultrasound diagnostic apparatus 1 includes an ultrasound diagnostic apparatus main body 10, an ultrasound probe 20 and an external battery unit 30.

The ultrasound probe 20 transmits ultrasound waves (transmission ultrasound waves) to the subject under examination not shown in the drawings, and receives reflected waves (reflected ultrasound waves (including scattered ultrasound waves)) of the ultrasound waves reflected at this subject under examination. The ultrasound diagnostic apparatus main body 10 is connected to the ultrasound probe 20 via a cable 21. The ultrasound diagnostic apparatus main body 10 causes the ultrasound probe 20 to transmit the transmission ultrasound waves to the subject under examination by transmitting a drive signal of an electrical signal to the ultrasound probe 20, and performs imaging of the internal state of the subject under examination as an ultrasound image on the basis of the received signals which are electrical signals generated in the ultrasound probe 20 according to the reflected ultrasound waves from inside the subject under examination received by the ultrasound probe 20.

In an example shown in FIG. 1, the ultrasound diagnostic apparatus main body 10 includes a display monitor 11 for displaying the ultrasound image and the like, an operation button 12 and the like on the front surface. The main body housing 13 is made of metal (for example, made by magnesium die casting), and has a folding stand 14 on the back surface so as to be placed on a table or the like to be used.

The folding stand 14 is formed in a U-shape. The mounting portion of the battery unit 30 is provided on the main body back surface in the range surrounded when the folding stand 14 is folded. FIG. 1 shows a state in which the battery unit 30 is actually mounted.

The battery unit 30 is externally attached to the ultrasound diagnostic apparatus main body 10 as shown in FIG. 1, that is, integrally attached to the ultrasound diagnostic apparatus main body 10 so as to be arranged in the external space of the ultrasound diagnostic apparatus main body 10.

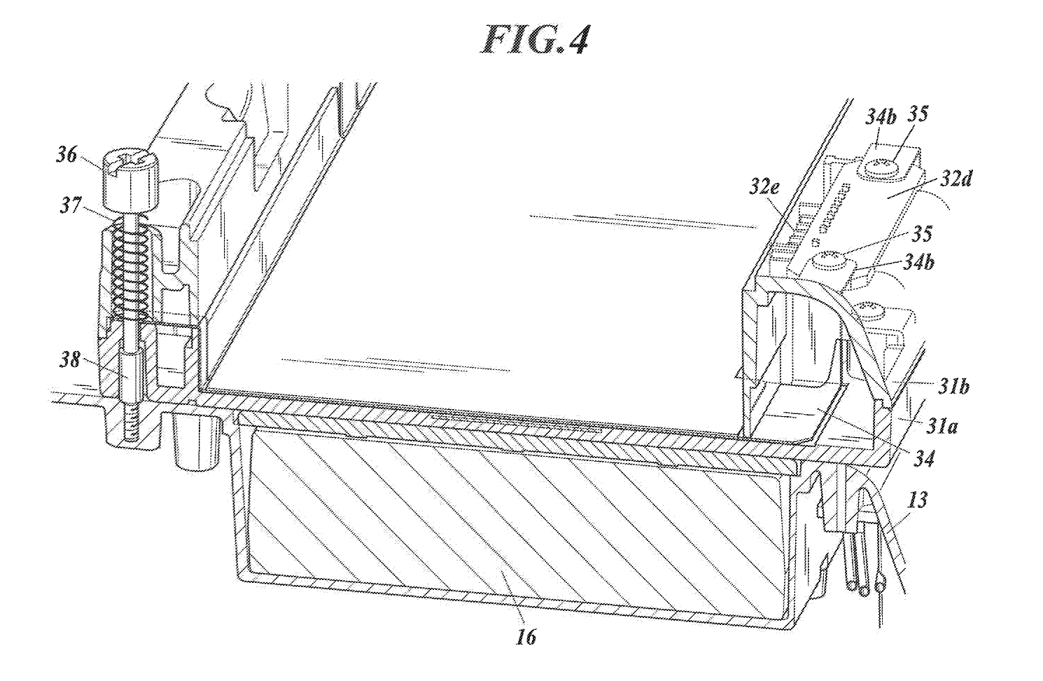

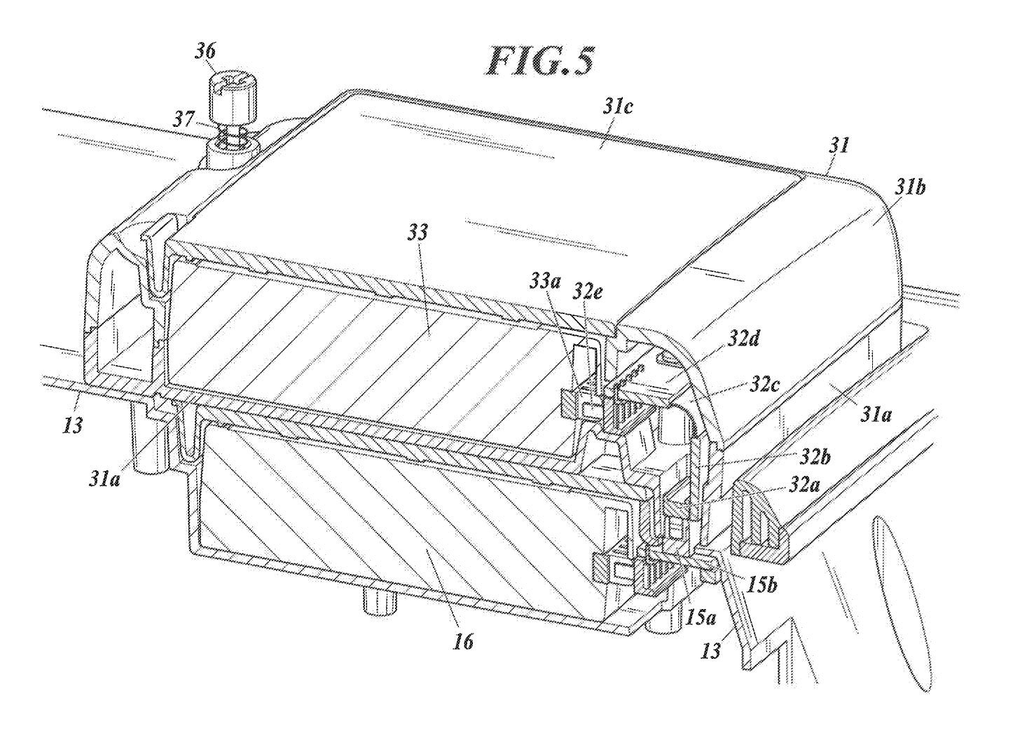

The battery unit 30 supplies electric power to the ultrasound diagnostic apparatus main body 10. In the embodiment, the ultrasound diagnostic apparatus main body 10 includes an internal battery 16 (see FIGS. 4 and 5). However, the ultrasound diagnostic apparatus main body 10 may not include the internal battery so that the ultrasound diagnostic apparatus main body 10 is used by battery drive only when the external battery unit 30 is attached.

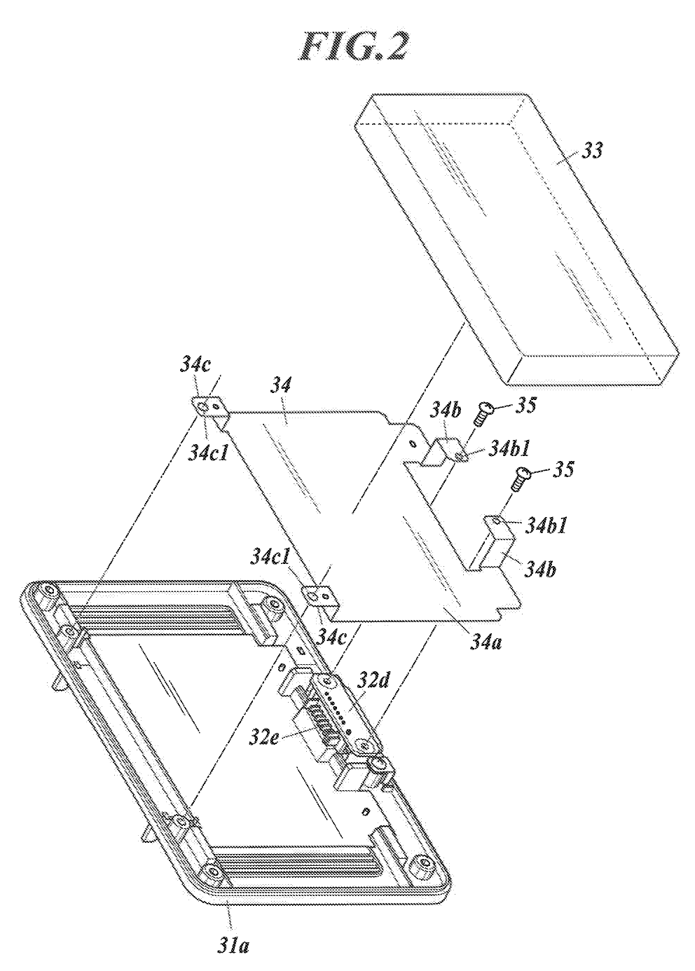

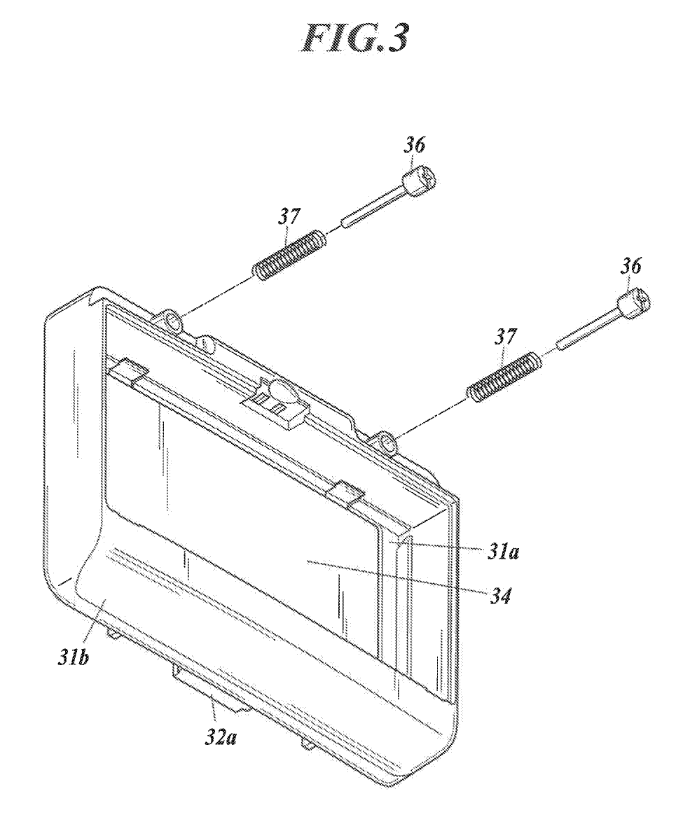

FIGS. 2 and 3 show exploded views of the battery unit 30. FIGS. 4 and 5 show sectional views in a state in which the battery unit 30 is attached to the ultrasound diagnostic apparatus main body 10.

The battery unit 30 has a resin battery housing 31 formed of parts 31a, 31b and 31c. The battery housing 31 is configured in a box shape by the plate-like part 31a on the attachment side, the frame-like part 31b which is connected to the peripheral portion of the part 31a and surrounds the battery 33, and the cover part 31c which covers the opening on the back side of the part 31b. The battery housing 31 contains the battery 33 therein.

An electrical connector 32a for connection to the ultrasound diagnostic apparatus main body 10 is fixed to the part 31a of the battery housing 31 via the wiring substrate 32b as shown in FIG. 5. A wiring substrate 32d to be connected to the wiring substrate 32b via a flexible wiring substrate 32c is similarly fixed to the part 31a of the battery housing 31. A connection terminal 32e of the battery 33 is mounted on the wiring substrate 32d, and connected to a connection terminal 33a provided to the battery 33, so as to be fit each other. As shown in FIG. 5, in a state in which the battery unit 30 is attached to the ultrasound diagnostic apparatus main body 10, the wiring substrate 32b is arranged in a direction orthogonal to the back surface of the ultrasound diagnostic apparatus main body 10, the wiring substrate 32d is arranged in a direction parallel to the back surface, and thus the arrangement is made by a relative angle of 90 degrees. This is because the connection direction of the electrical connector 32a to the ultrasound diagnostic apparatus main body 10 and the connection direction of the connection terminal 32e to the battery 33 are different from each other by 90 degrees.

As shown in FIG. 2, the conductive member 34 is arranged between the battery 33 and the internal wall (part 31a) of the battery housing 31. The conductive member 34 is formed to be a plate-like member or a sheet-like member so as to increase the capacity of the conductive member 34 without making the space inside the battery housing 31 tight. In the conductive member 34, a main part 34a having the largest area is arranged along the surface of the battery 33 and the internal wall (part 31a) of the battery housing 31 so as to be arranged between the battery 33 and the internal wall of the battery housing 31.

Connection parts 34b, 34b are provided so as to extend from the main part 34a of the conductive member 34. Holes 34b1 are formed at the ends of the connection parts 34b, 34b, and set screws 35 can be inserted through the holes 34b1. The set screws 35 are inserted through the holes 34b1 and holes provided on the wiring substrate 32d and screwed to female screws formed in the part 31a, and the conductive member 34 and the wiring substrate 32d are joined to each other. The connection parts 34b is pressed against the ground pattern formed on the wiring substrate 32d via the set screws 35, and electrically connected.

The ground pattern formed on the wiring substrate 32d functions as a ground by being connected to a conductive main body housing 13 via the wiring pattern in the flexible wiring substrate 32c, the wiring pattern in the wiring substrate 32b, the terminal in the electrical connector 32a, the terminal of an electrical connector 15a in the ultrasound diagnostic apparatus main body 10 shown in FIG. 5, and a wiring substrate 15b of the ultrasound diagnostic apparatus main body 10.

As described above, the conductive member 34 is grounded to the ultrasound diagnostic apparatus main body 10. The conductive member 34 is grounded to the ultrasound diagnostic apparatus main body 10 via the electrical connector 32a of the battery unit 30.

The conductive member 34 is also grounded to the ultrasound diagnostic apparatus main body 10 via the following path.

Connection parts 34c, 34c are extended from the main part 34a of the conductive member 34. Holes 34c1 are formed at the ends of the connection parts 34c, 34c. Fixing bolts 36 shown in FIGS. 3 to 5 can be inserted through the holes 34c1, and the inner diameter of the hole 34c1 is smaller than the outer diameter of a coil spring 37. The fixing bolts 36 are inserted through the holes 34c1 and the holes provided in the parts 31a and 31b, and screwed so as to be joined to female screws formed in the conductive main body housing 13 via joint screws 38. The fixing bolts 36 are inserted through the coil springs 37 and the holes 34c1 provided in the conductive member 34, and the coil springs 37 are pressed against the heads of the fixing bolts 36 and the conductive member 34 around the holes 34c1. Thereby, the conductive member 34 is electrically conducted to the fixing bolts 36. The connection part 34c is sandwiched between the part 31a and the part 31b, and not exposed outside. Though the part 31a which is an insulating material intervenes between the heads of the fixing bolts 36 and the connection parts 34c, electrical connection is made by the coil springs 37.

As described above, the conductive member 34 is grounded to the ultrasound diagnostic apparatus main body 10 via the bolts 36, 38 for fixing the battery housing 31 to the ultrasound diagnostic apparatus main body 10. The fixing bolts 36 may be directly screwed so as to be joined to the main body housing 13 without using the joint screws 38.

### Effect, Substantiative Measurement

According to the ultrasound diagnostic apparatus 1 in the embodiment, in a case where the housing 31 of the battery unit 30 which is externally attached to the ultrasound diagnostic apparatus main body 10 is a resin housing, it is possible to enhance grounding of the battery unit 30, reduce electromagnetic radiation from the battery unit 30 and improve the EMC performance.

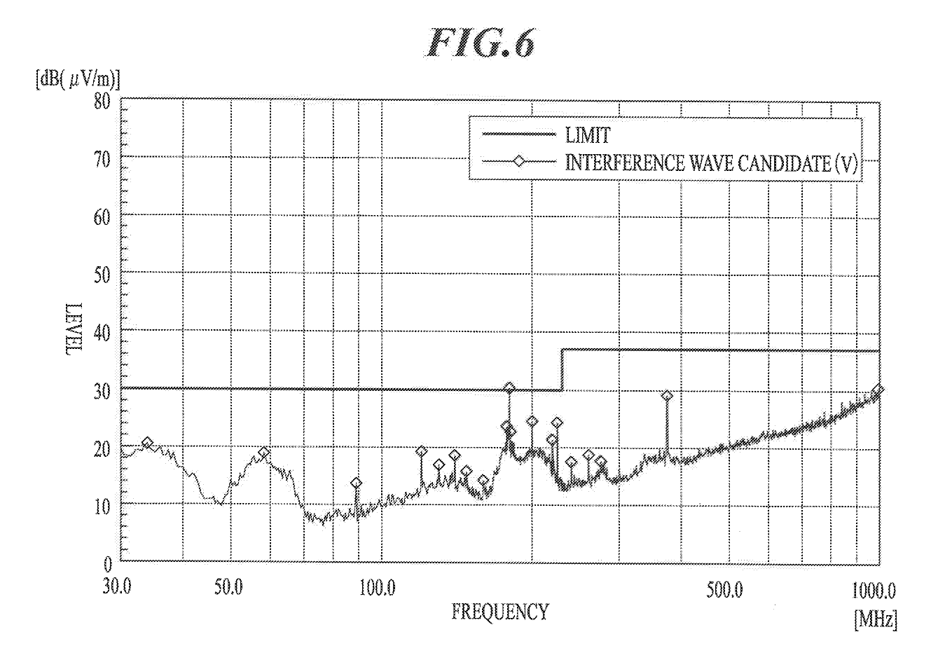

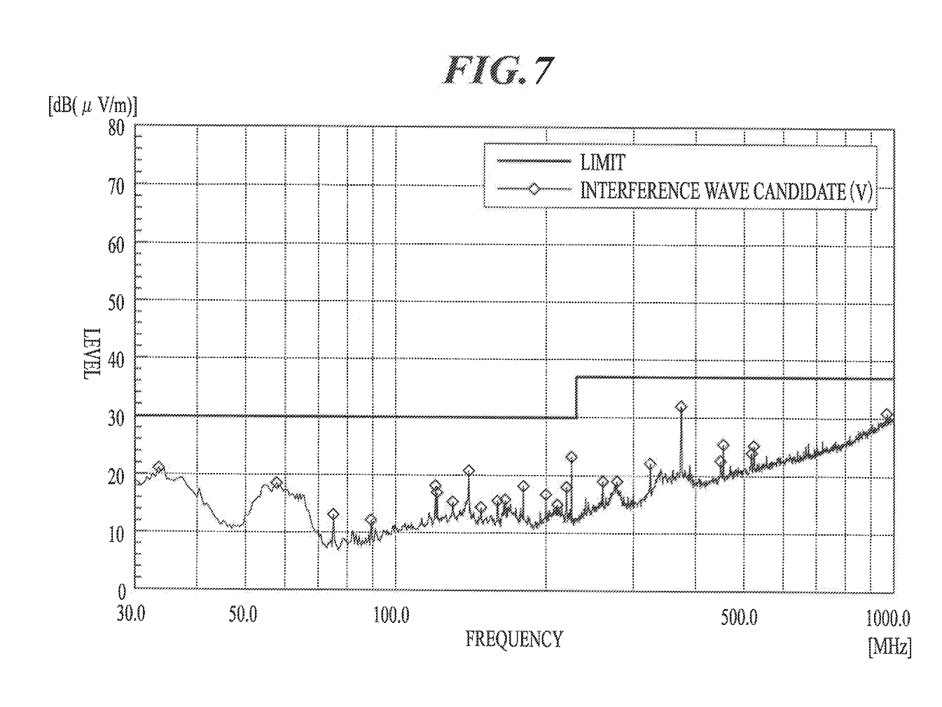

FIG. 6 is a graph showing frequency distribution of the result obtained by measuring the electromagnetic radiation amount of an ultrasound diagnostic apparatus according to a comparative example. FIG. 7 is a graph showing frequency distribution of the result obtained by measuring the electromagnetic radiation amount of an ultrasound diagnostic apparatus according to an example of the present invention.

The example of the present invention has the configuration according to the above-described embodiment. The comparative example has the configuration corresponding to the configuration excluding the conductive member 34 from the example of the present invention. In both of the examples, the measured position is on the main body back surface side and located around the electrical connection portion between the ultrasound diagnostic apparatus main body and (the battery 33 of) the external battery unit. Both of the examples have a common operation situation in which the ultrasound diagnostic apparatus main body is connected to the AC power source and (the battery 33 of) the external battery unit and the internal battery (16) are charged for use.

FIGS. 6 and 7 show a limit line of the electromagnetic interference wave applied in medical facilities.

As shown in FIG. 6, in the comparative example, an interference wave candidate exceeding the limit line appeared among the interference wave candidates which are prominent peaks.

As shown in FIG. 7, in the example of the present invention, it was possible to suppress all the interference wave candidates to be lower than the limit line. The factor of this is considered that the conductive member (34) arranged in the external battery unit is grounded and the ground potential of the battery unit is stabilized. Especially, the factor is considered that the grounding is enhanced by not only the connection via the electrical connector 32a but also by applying the fixing bolts 36 as the ground path in the example of the present invention as described in the above embodiment.

### Others

In the above embodiment, the conductive member 34 arranged between the battery 33 and the internal wall of the battery housing 31 is grounded to the ultrasound diagnostic apparatus main body 10 via the electrical connector 32a, and grounded to the ultrasound diagnostic apparatus main body 10 also via the fixing bolts 36. However, the grounding may be performed by only one of the paths of the electrical connector 32a and the fixing bolts 36.

The conductive member 34 may be grounded to the ultrasound diagnostic apparatus main body 10 by connection via other paths. For example, when the conductive member 34 is partially exposed toward the outer surface side of the part 31a as a contact terminal portion to the main body housing 13 and the battery unit 30 is fixed to the main body housing 13 with the fixing bolts 36, there can be a configuration such that the contact terminal portion is connected to the main body housing 13 by being pressed against the main body housing 13. In such a case, it is possible to enhance water-tightness by dividing the part corresponding to the part 31a into two parts so that the wall portion to set the conductive member 34 in the part 31a has a double-walled structure and performing bonding and sealing so as to sandwich the conductive member 34 between the double walls.

As for detailed configurations and detailed operations of the components forming the ultrasound diagnostic apparatus 1 in the above embodiment, modifications can be performed as needed within the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-006820, filed on Jan. 19, 2018, is incorporated herein by reference in its entirety.

What is claimed is:

1. An ultrasound diagnostic apparatus, comprising:
an ultrasound diagnostic apparatus main body which transmits a drive signal to an ultrasound probe that transmits and receives ultrasound waves, and which generates an ultrasound image based on a received signal that is obtained from the ultrasound probe; and
a battery unit which is externally attached to the ultrasound diagnostic apparatus main body and supplies electric power to the ultrasound diagnostic apparatus main body, wherein
the battery unit includes:
a battery housing which is made of resin and contains a battery therein;
an electrical connector for connection to the ultrasound diagnostic apparatus main body, the electrical connector being fixed to the battery housing; and
a conductive member which is arranged between the battery and an inner wall of the battery housing, and
the conductive member is grounded to the ultrasound diagnostic apparatus main body.

2. The ultrasound diagnostic apparatus according to claim 1, wherein the conductive member is grounded to the ultrasound diagnostic apparatus main body via the electrical connector.

3. The ultrasound diagnostic apparatus according to claim 1, wherein the conductive member is grounded to the ultrasound diagnostic apparatus main body via a bolt for fixing the battery housing to the ultrasound diagnostic apparatus main body.

4. The ultrasound diagnostic apparatus according to claim 3, wherein the conductive member and the bolt are electrically conducted to each other by inserting the bolt through a hole provided in the conductive member and a coil spring, and causing the coil spring to be pressed against a head of the bolt and the conductive member around the hole.

5. The ultrasound diagnostic apparatus according to claim 3, wherein a main body housing which is a housing of the ultrasound diagnostic apparatus main body is conductive, and the conductive member is grounded to the ultrasound diagnostic apparatus main body by joining the bolt to the main body housing.

6. The ultrasound diagnostic apparatus according to claim 1, wherein the conductive member is a plate-like member or a sheet-like member.

7. An external battery unit of an ultrasound diagnostic apparatus, the battery unit being externally attached to an ultrasound diagnostic apparatus main body and supplying electric power to the ultrasound diagnostic apparatus main body which transmits a drive signal to an ultrasound probe that transmits and receives ultrasound waves, and which generates an ultrasound image based on a received signal that is obtained from the ultrasound probe, and the battery unit comprising:
a battery housing which is made of resin and contains a battery therein;
an electrical connector for connection to the ultrasound diagnostic apparatus main body, the electrical connector being fixed to the battery housing; and
a conductive member which is arranged between the battery and an inner wall of the battery housing, wherein
the conductive member is grounded to the ultrasound diagnostic apparatus main body.

8. The external battery unit of the ultrasound diagnostic apparatus according to claim 7, wherein the conductive member is grounded to the ultrasound diagnostic apparatus main body via the electrical connector.

9. The external battery unit of the ultrasound diagnostic apparatus according to claim 7, wherein the conductive member is grounded to the ultrasound diagnostic apparatus main body via a bolt for fixing the battery housing to the ultrasound diagnostic apparatus main body.

10. The external battery unit of the ultrasound diagnostic apparatus according to claim 9, wherein the conductive member and the bolt are electrically conducted to each other by inserting the bolt through a hole provided in the conductive member and a coil spring, and causing the coil spring to be pressed against a head of the bolt and the conductive member around the hole.

11. The external battery unit of the ultrasound diagnostic apparatus according to claim 9, wherein the conductive member is grounded to the ultrasound diagnostic apparatus main body by joining the bolt to a main body housing which is a housing of the ultrasound diagnostic apparatus main body and which is conductive.

12. The external battery unit of the ultrasound diagnostic apparatus according to claim 7, wherein the conductive member is a plate-like member or a sheet-like member.
